(19) United States
(12) Patent Application Publication
(10) Pub. No.: US 2015/0313699 A1
(43) Pub. Date: Nov. 5, 2015

(54) MUSCLE CONDITIONING DEVICE

(71) Applicant: UCL BUSINESS PLC, London (GB)

(72) Inventors: Richard DAY, Esher (GB); Quentin PANKHURST, St. Albans (GB)

(21) Appl. No.: 14/651,536

(22) PCT Filed: Dec. 10, 2013
(86) PCT No.: PCT/GB2013/000541

§ 371 Date: Jun. 11, 2015

(30) Foreign Application Priority Data
Dec. 11, 2012 (GB) 1222255.0

(51) Int. Cl. A61F 2/00

(52) U.S. Cl. CPC A61F 2/0018; A61F 2210/009; A61F 2250/0001

(57) ABSTRACT

The present invention relates to a muscle conditioning device comprising one or more implantable bodies and an actuator, where in use, the implantable bodies are magnetically coupled to the actuator and are resiliently positionable to administer mechanical strain to the muscle on operation of the actuator. It also relates to individual elements of the device; to compositions and kits thereof; and to uses of and methods involving the device.

### FIELD OF INVENTION

The invention relates to a muscle conditioning device which can be used to promote cell growth.

### BACKGROUND TO THE INVENTION

Faecal incontinence remains a common affliction associated with marked social and psychological disability. There are many causes, so comprehensive assessment is paramount. Treatment strategies are aimed at reducing the burden of incontinence so that quality of life is improved and, if definitive treatment is not possible, at helping patients cope with their symptoms.

Therapeutic strategies are varied and dependent on local expertise and available facilities. Techniques currently available to help treat this condition include sacral nerve stimulation and invasive surgical procedures. The colostomy, once thought to be the last resort, is also increasingly regarded as offering hope for some of the more severely troubled patients.

However, these techniques have distinct disadvantages. Sacral nerve stimulation requires expensive evaluations of peripheral nerve tissue and involves the use of low-level electrical stimulation to stimulate muscle movement. Bulking materials have been used to enlarge the sphincter muscles and thereby improve the seal created by the muscle, however these materials often migrate to other regions of the body. Further, even when fixed in place, there is often no change in the resting or squeeze pressures exerted by the muscle.

Surgical procedures, such as sphincteroplasty, dynamic graciloplasty, artificial bowel sphincter and permanent end stoma have been used. However, these procedures are not only complex and expensive but also often cause complications including infections, evacuation difficulties and chronic pain. The colostomy is a complex and invasive procedure and potentially involves the removal of functional, healthy digestive tract and forces the patient to rely on a colostomy bag merely due to a single malfunctioning sphincter.

Attempts have been made to utilize the elementary forces that occur between magnets for treating faecal incontinence. Magnets are surgically implanted around the anal sphincter and the magnetic attraction between the magnets closes the anal canal creating a barrier to prevent involuntary loss of stool. The magnetic bond is temporarily broken to allow the voluntary passage of stool and restored immediately thereafter. Many of these systems seek to replace the role of the sphincter and do not restore the original sphincters function.

The inventors have discovered, while seeking to address these problems, a novel system for conditioning muscles. This device can be used in conjunction with a wide range of sphincters and other types of muscle, such as, for example the pyloric sphincter or muscles affected by conditions like muscular dystrophy.

The invention is intended to overcome or ameliorate at least some of the problems associated with the above mentioned solutions.

### SUMMARY OF THE INVENTION

In a first aspect of the invention, there is provided a muscle conditioning device comprising one or more implantable bodies and an actuator, where in use, the implantable bodies are coupled to the actuator and are resiliently positionable to administer mechanical strain to the muscle on operation of the actuator. Movement of the implantable bodies causes mechanical strain on the surrounding muscle tissue. This strain, among other effects, promotes an increase in cell proliferation and has also been found to condition the muscle tissue to which the strain is applied which improves the ability of the tissue to successfully accept cell grafts.

The device is typically used with involuntary or smooth muscles. This type of muscle tissue is not directly capable of being exercised by an individual. However, the device may also be used with voluntary muscles. For example, elderly individuals, patients suffering from muscular dystophy or sarcopenia suffers may have difficulty in exercising their muscles through conventional means. As such, the device allows direct application of mechanical strain to the tissue without requiring exertion by the patient.

Typically, the device is a sphincter conditioning device and even more typically an anal sphincter conditioning device. The term ‘conditioning’ is intended to mean an improvement in the function of the sphincter for example by promoting cell growth (hyperplasia), increasing expression of contractile protein apparatus, treating a muscle to better uptake grafted cells and/or causing cell hypertrophy. Most sphincters are involuntary muscles and therefore can not be controlled at an individuals will. Accordingly, the device is typically used in conditioning sphincters. Where the muscle is a sphincter, the mechanical strain provided to the muscle is typically a repeated strain meaning that the force applied to the implantable bodies is an oscillating force. Even more typically, the strain is applied in a cyclical or arc-like manner, i.e. the force is applied clockwise and/or anticlockwise substantially around the circumference of the sphincter.

The term, “resiliently positionable” is intended to mean that when the implantable bodies are positioned within a target tissue in a resting position and a force is applied to move the implantable bodies away from said resting position, the implantable bodies are urged back towards the resting position due to the resistance of the tissue into which the implantable bodies have been implantable.

In one embodiment of the invention, the implantable bodies and the actuator may be magnetically coupled. The term “magnetically coupled” is intended to mean that there is a magnetic interaction, either repulsion or attraction, between the implantable bodies and the actuator such that when the actuator is brought into close proximity with the implantable bodies a force is exerted on the implantable bodies causing movement of the implantable bodies. When the actuator is removed or the magnetic field provided by the actuator is removed the implantable bodies are then resiliently returned to their initial position due to the turgidity of the tissue into which they are implanted. As magnetic fields can easily permeate through tissue, using magnetic fields to move the implantable bodies allows manipulation of the implantable bodies without requiring regular invasive procedures or the need to maintain open channels into muscle tissue which could become blocked or infected.

Typically, the implantable bodies are magnetic and the actuator comprises at least one magnet. The term “magnetic material” is intended to mean any material that is either magnetically hard or magnetically soft. Typically the magnetic material will be easy to magnetise or is a magnetically soft material. If the implantable bodies were to be magnets (i.e. hard magnetic materials or permanent magnets) the magnetic field generated by these magnets would not be easy to change and the direction of the field would depend on the orientation of the implantable bodies. Therefore, in order to modify the magnetic field it would be necessary to remove the implantable bodies, alter the magnetic properties and reinsert them. Further, as the actuator is not implanted into the body, the size and shape of the magnets used can be more varied if included as part of the actuator. The at least one magnet is typically a permanent magnet which may comprise iron and even more typically comprises neodymium. Typically the at last one magnet is a neodymium iron born (NdFeB) permanent magnet. Permanent magnets are often preferred to electromagnets as they are cheap to manufacture and incorporate into a device.

The implantable bodies may be intended to remain in situ or to be removed or to be biodegradable such that they break down after some time and are absorbed or excreted. When the implantable bodies remain in situ, they may remain permanently or substantially permanently magnetisable or be permanent magnets. Alternatively, the magnets or magnetism may actively or passively degrade or reduce over time.

The at least one magnet may be an electromagnet. This allows the strength of the magnetic field to be controlled by changing the current passing through the magnet. The size and strength of muscles in different patients will vary between individuals and these parameters will also change with use of the device over time. Accordingly, the magnetic field required to induce the mechanical strain necessary to achieve optimal strength enhancement will change over time. Therefore, using an electromagnet with the actuator allows simple, fine tuning of the magnetic field and corresponding force applied to the implantable bodies to provide optimal mechanical strain to the target muscle.

It is often the case that the magnetic material is selected from: iron oxide, ferrites, rare-earth metals, titanium coated neodymium iron boron, samarium cobalt and magnetic steels or a combination thereof. Typically, the implantable bodies are magnetic at room temperature or above and even more typically, super paramagnetic at room temperature or above. This means that the implantable bodies do not retain any magnetisation after removal of a magnetic field, Accordingly, the implantable bodies are free-flowing which aids minimally invasive delivery of the implantable bodies through an instrument such as a syringe or needle but retain a strong response when exposed to a magnetic field.

Typically, the implantable bodies comprise iron oxide. Iron oxide is a relatively cheap ferromagnetic material and is less toxic than the nickel or cobalt equivalents.

In another embodiment of the invention, the implantable bodies may comprise an inert coating or inert material. Incorporating an inert material into either the bulk of an implantable body or as a coating on the surface of an implantable body prevents the implantable bodies from degrading or being broken down and assimilated into the body. The term “inert” is intended to mean that something does not react or break down, or negligibly reacts or is broken down, when incorporated into the body. Inert coatings are also useful to prevent oxidation or other chemical interaction of the magnetic materials in vivo, especially when the implantable bodies are very small. The inert coating or material is often selected from: gold, titanium, silicone, biocompatible glass, biocompatible polymers including polyesters, polyethylene glycol or a combination thereof. Typically, the implantable bodies comprise yttria-alumina-silica (YAS) glass.

The implantable bodies may be rounded and may also have a smooth surface. This improves comfort for the user as the implantable bodies do not include sharp protrusions or vertices. The implantable bodies may however have a roughened or uneven surface to help keep the implantable bodies in place attached to the target tissue and prevent migration to other regions of the body. The implantable bodies may also comprise an adhesive coating to further prevent migration of the implantable bodies. Typically, the devices are ovoid or even more typically, substantially spherical. These shapes not only improve user comfort but also have a packing parameter that encourages the formation of channels between the implantable bodies. This allows cell growth in between the implantable bodies and thereby further helps the target tissue to hold the implantable bodies resiliently in position.

The diameter of the implantable bodies is usually in the range of 1 nm to 1 mm. Alternatively, the implantable bodies may be nanoparticles and the nanoparticles are typically positionable within or on the surface of cells of a human or animal body. This allows mechanical strain to be applied to individual cells and promote a more even distribution of mechanical strain across an area of muscle tissue. The nanoparticles may be delivered to cells in vitro prior to delivery as part of a cell therapy procedure. After delivery in vivo, the loaded cells may also be conditioned with an external magnetic field to facilitate cell engraftment and differentiation.

In a further embodiment, the nanoparticles may comprise a targeting moiety adapted to deliver nanoparticles to a specific cell type. This allows nanoparticles to be delivered to specific cell types and improves the ease and number of techniques that can be used to administer the nanoparticles to the cells. The term “nanoparticle” is intended to refer to particles having a diameter in the range 1 nm to <1 μm.

The implantable bodies may also be larger than this having a diameter in the range 1 μm to 1 mm. These implantable bodies are too large to be incorporated into cells but can be positioned in and around specific areas of muscle tissue. These implantable bodies are easier to manufacture and can be monitored more easily using techniques known to the person skilled in the art. Further, implantable bodies within this range can be manufactured out of a wider range of materials using simpler techniques. The implantable bodies may for instance be made from inert materials, such as polymers or glasses, doped with magnetic particles.

Although the implantable bodies may be made from biologically inert materials which are not broken down by the body, the implantable bodies may also be made from biodegradable and/or bio-assimilable materials. This allows the implantable bodies to break down over time and be removed from the body without the need for an invasive surgical removal procedure. Typically, the composition of these implantable bodies will be such that the rate of break down of the implantable bodies causes the implantable bodies to be removed from the body, after a patient has undergone a scheme of treatment with the invention.

Typically, the actuator used in the present invention is operable to generate an magnetic field which creates an oscillating magnetic force on the implantable bodies. The provision of a changing magnetic force causes the implantable bodies to move away from a resting position against the bias provided by the muscle before being urged back towards the resting position by the resilience of the muscle tissue. An oscillating magnetic force provides periodic mechanical strain to the muscle thereby promoting cell proliferation. Further, it has also been found by the inventors that cell phenotype can be modified by varying the rate of oscillation of the force and the strength of the magnetic field.

In one embodiment of the invention, the actuator may further comprise an elongate body and a rotatable member, wherein the rotatable member is located within the elongate body and is connected to at least one magnet. Mounting the at least one magnet on a rotatable member allows the oscillating magnetic force applied to the implantable bodies to be controlled by varying the speed of rotation. The at least one magnet is encased within an elongate body to prevent the rotatable member from catching on parts of the body and to ensure that the mechanism is not obstructed. Alternatively, the at least one magnet may be mounted on an extendible arm located within the elongate body arranged to move along the axis of the elongate body.

The rotatable member may be rotatable about the axis of the elongate body and even more typically, the at least one magnet is arranged such that the magnetic field projects radially outwards relative to the axis of rotation. Arranging the magnet such that the magnetic field projects radially outwards, is particularly useful as the magnetic field propagates in a radial arc outward from the walls of the elongate body. This allows the device to influence implantable bodies located deeper within the body than with other orientations and provides a strong force on the implantable bodies followed by relaxation as the rotatable member rotates.

It is often the case that the actuator may further comprise a magnetically soft flux concentrator. Typically, this is a layer of soft steel between the at least one magnet and the rotatable member. The term “soft steel” is intended to mean magnetically soft. The layer of soft steel helps to ‘throw’ the magnetic field propagating inward toward the rotating member, generated by the magnet, towards the ends of the actuator. This ensures that there is effectively a zero field except for the arc propagating outward from the magnet in a radial manner. This helps to ensure that the implantable bodies experience a strong pull and relaxation as the magnetic field effectively only propagates from one side of the magnet as the rotatable member rotates.

Typically the actuator further comprises a drive means arranged to rotate the rotatable member and even more typically, the actuator further comprises a power supply to power the drive means. This may be through connection to the mains or using a battery.

The actuator may also further comprise a removable end cap at the distal end of the actuator. The replaceable end cap can be disposed of and changed to allow multiple users to use the same actuator hygienically. Typically, the actuator further comprises a handle portion at the proximal end of the actuator. This makes the device ergonomic and easier to use. The handle portion may also comprise a shoulder arranged to prevent the actuator from being over inserted into the body.

In alternative embodiments, the oscillating magnetic force created on the implantable bodies due to the magnetic field of the actuator may be generated not by having the magnet rotate, but by having it move, for example in a linear fashion, between first and second positions, such that the magnetic force applied to the implantable bodies will change according to the position of the magnet. This could be linear movement along the length of the elongate member between the distal end of the device and the proximal end of the device. Alternatively, the magnetic force applied to the implantable bodies may be altered simply by altering the strength of the magnetic field or even by turning it on and off.

In alternative embodiments, the actuator may take a different form to that described above. It may still be for insertion into a body cavity, for example, or could be for application to the body externally, for example forming part of a chest strap for use in applying a magnetic force to the pyloric sphincter or a seat/saddle for use in applying force to the anal sphincter. It could take the form of a plate to be placed over an area of muscle to be treated. The form of the actuator may be determined by the area of the body with which it is to be used.

In a second aspect of the invention, there is provided a non-totipotent cell comprising the one or more implantable bodies, wherein the one or more implantable bodies are magnetic nanoparticles. The implantable bodies can be administered into cells ex vivo and then the cells can be administered to the target muscle tissue. These cells may be muscle cells which will graft to the muscle tissue being conditioned or pluripotent stem cells which will form new muscle tissue and become incorporated into the existing muscle thereby further conditioning the muscle. The cells used with the present invention are not capable of forming a human being.

In a third aspect of the invention, there is provided a composition comprising the cells according to the second aspect of the invention and a pharmaceutically acceptable excipient.

In a fourth aspect of the invention, there is provided a composition comprising one or more implantable bodies and a pharmaceutically acceptable excipient, wherein the one or more implantable bodies are magnetic nanoparticles comprising a targeting moiety adapted to deliver the nanoparticles to a specific cell type.

In a fifth aspect of the invention, there is provided a composition comprising one or more implantable bodies and a pharmaceutically acceptable excipient, wherein the implantable bodies have a diameter in the range of 1 μm to 1 mm and comprise a magnetic material and an inert coating or inert material. The inert coating or material is often selected from: gold, titanium, silicone, biocompatible glass, biocompatible polymers including polyesters, polyethylene glycol or a combination thereof. Typically, the implantable bodies comprise yttria-alumina-silica (YAS) glass. It is often the case that the magnetic material is selected from: iron oxide, ferrites, rare-earth metals, titanium coated neodymium iron boron, samarium cobalt and magnetic steels. Typically, the implantable bodies are super paramagnetic at room temperature or above. Typically, the implantable bodies comprise iron oxide.

The implantable bodies and/or cells according to any of the first, second, third, fourth and fifth aspects of the invention are typically stored in an excipient in order to maintain the implantable bodies and/or cells in a sterile condition and improve delivery. The excipient used in both the second, third, fourth and fifth aspects of the invention typically has a viscosity sufficient to form a homogeneous suspension with the implantable bodies and/or cells. The term ‘homogeneous suspension’ is intended to mean a stable mixture of implantable bodies and/or cells in a medium, wherein the implantable bodies and/or cells are uniformly dispersed throughout the medium. Using a viscous medium, maintains a stable and uniform distribution of the implantable bodies and/or cells which allows for accurate delivery of the implantable bodies and/or cells and minimises sedimentation and creaming. Where the magnetic material used in the implantable bodies is iron oxide, it is usually the case that the excipient will have a density of 5 g cm⁻³.

The excipient is typically a solution of polymeric material and may also be a hydrogel. The excipient may further comprise a sterilising agent to reduce the risk of infection and may also comprise an anti-inflammatory to reduce any swelling resulting from the implantation procedure. The excipient may also comprise an anaesthetic to minimise discomfort during the implantation procedure. The excipient is also typically non-toxic and biocompatible. Therefore, once the implantable bodies have been implanted, the excipient is capable of being absorbed by the body, broken down and excreted.

In a sixth aspect of the invention, there is provided a kit comprising the composition according to the second, third, fourth or fifth aspects of the invention and an applicator arranged to implant the one or more implantable bodies. It is often the case that the applicator may be a syringe or cannular. However, the applicator may also be an endoscopic or laproscopic type instrument. A variety of muscle groups can be influenced using the device and in order to deliver the implantable bodies and/or cells to different areas of the body, different instruments are required. Accordingly, the implantable bodies and/or can be implanted during endoscopic or laproscopic surgical techniques where deliver with a typical syringe is not possible.

In a seventh aspect of the invention there is provided, an actuator operable to generate an magnetic field to create an oscillating magnetic force on an implantable, comprising an elongate body, a rotatable member comprising at least one magnet and a magnetically soft flux concentrator between the at least one magnet and the rotatable member, wherein the rotatable member is located within the elongate body and is rotatable about the axis of the elongate body and the at least one magnet is arranged such that the magnetic field projects radially outwards relative to the axis of rotation.

In an eighth aspect of the invention, there is provided a device according to the first aspect of the invention for use in the treatment and/or prevention of faecal incontinence. The term, ‘faecal incontinence’ is intended to refer to weakness of the anal sphincter, one of the results of which is failure to control of the excretion of faecal matter. However, this term is also intended to refer to damage or general weakness of the sphincter. Where the sphincter has been weakened, for example as a result of trauma, the patient has an increased likelihood of developing complete faecal incontinence. Accordingly, the device can be used to condition the sphincter and thereby prevent complete faecal incontinence from occurring.

The device may also be used in the treatment and/or prevention of other diseases such as: muscular dystrophy, reflux, dysphagia, sarcopenia and urinary incontinence.

In an ninth aspect of the invention, there is provided a method of conditioning a sphincter using the device according to the first aspect of the invention comprising the steps of:

1) positioning one or more implantable bodies to administer mechanical strain to the muscle on operation of the actuator;
2) positioning the actuator in communication with the implantable body; and
3) operating the actuator to administer mechanical strain to the muscle.

In a tenth aspect of the invention, there is provided a method of treating faecal incontinence using the device according to the first aspect of the invention comprising the steps of:

1) positioning the implantable bodies in the intersphincteric plane;
2) inserting the actuator into the anal cavity; and
3) operating the actuator to administer mechanical strain to the anal sphincter.

The implantable bodies are typically introduced into the intersphincteric plane using a syringe or cannular. Typically, the implantable bodies are distributed around the circumference of the intersphincteric plane rather than in a single localised area. Alternatively, where damage has been caused to the sphincter, the implantable bodies may be located adjacent to the damaged portion of the sphincter. This provides localised mechanical strain to the damaged portion of the ring to promote recovery. The actuator is inserted into the anal cavity and operated for a period of time which may be in the range of 1 minute to 3 hours, or typically 5 minutes to 1 hour, or even more typically 10 minutes to 30 minutes.

Unless otherwise stated each of the integers described in the invention may be used in combination with any other integer as would be understood by the person skilled in the art. Further, although all aspects of the invention preferably “comprise” the features described in relation to that aspect, it is specifically envisaged that they may “consist” or “consist essentially” of those features outlined in the claims. In addition, all terms, unless specifically defined herein, are intended to be given their commonly understood meaning in the art.

Further, in the discussion of the invention, unless stated to the contrary, the disclosure of alternative values for the upper or lower limit of the permitted range of a parameter, is to be construed as an implied statement that each intermediate value of said parameter, lying between the smaller and greater of the alternatives, is itself also disclosed as a possible value for the parameter.

In addition, unless otherwise stated, all numerical values appearing in this application are to be understood as being modified by the term “about”.

The invention will now be described, by way of example only, with reference to the accompanying figures:

### BRIEF DESCRIPTION OF THE DRAWINGS

FIG. 1a to 1e shows a diagrammatic cross section representation of the device in use with a sphincter.

FIG. 2 shows a part-cutaway view of the actuator of the invention.

FIG. 3 shows a side view of rotatable member.

FIG. 4 shows a cross-sectional view of rotatable member.

FIG. 5a 5b shows the results of subjecting human rectal smooth muscle cells (HRSMC) to mechanical strain.

FIG. 6 shows the magnetisation of the microspheres in one embodiment of the device.

FIG. 7 shows the displacement of ferromagnetic material on tissue (from 0.02 to 0.24 mm in displacement Y, as shown by the path of rotation of the magnet; up to 0.10 mm in displacement Z, with the maximum displacement indicated by the circle), caused by the rotation of the actuator of the invention.

FIG. 8 (a)-(d) shows the implantation and integration of microspheres between muscle planes.

### SPECIFIC DESCRIPTION

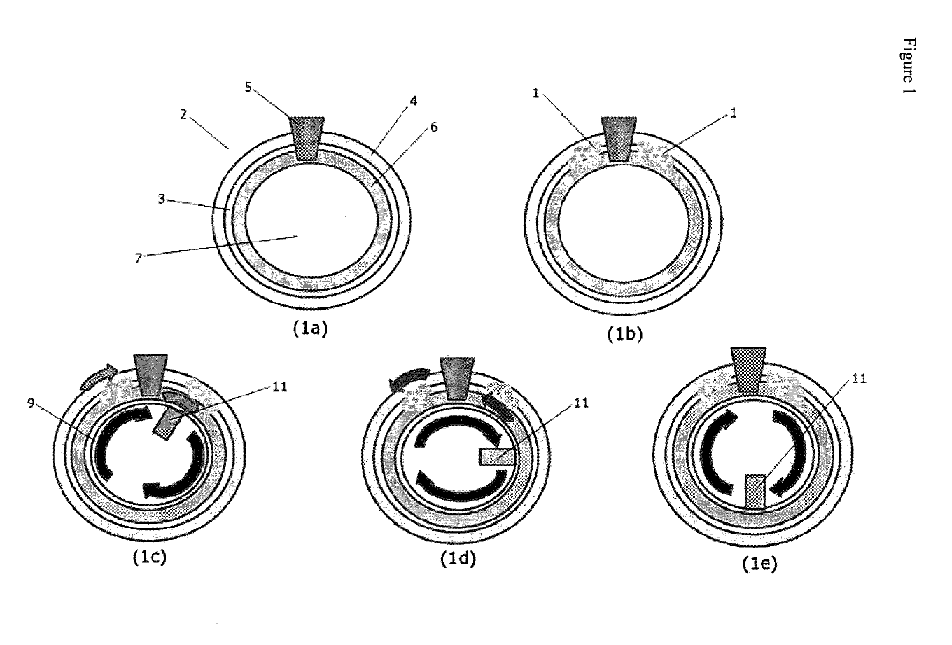

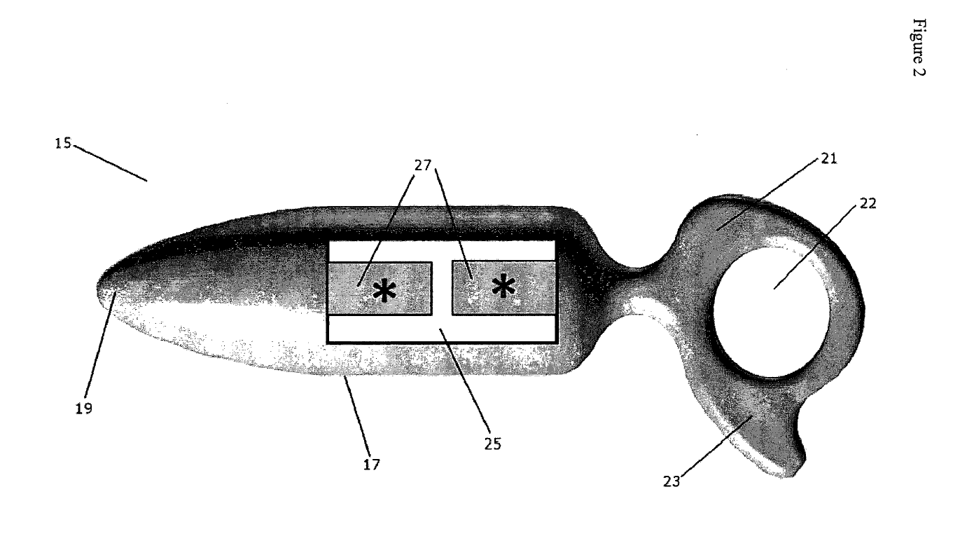

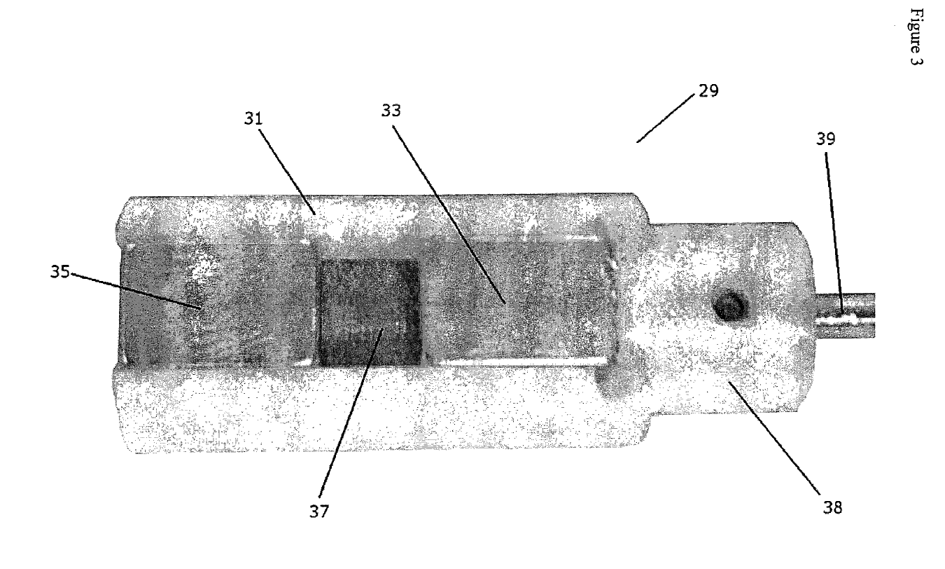

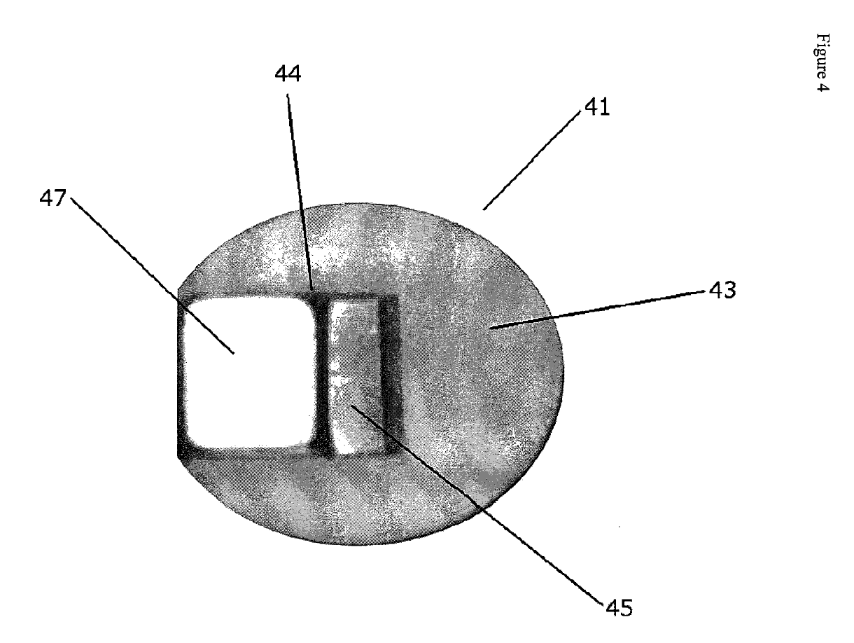

FIG. 1a shows a schematic, cross sectional representation of the anal sphincter 2 as a series of concentric rings representing from outside inward the external anal sphincter 4, the intersphincteric plane 3, the internal anal sphincter 6 and the anal cavity 7 in the centre. A damaged region of tissue 5 of the anal sphincter 2 is shown which spans the inner and outer anal sphincter 4, 6. FIG. 1b shows spherical, superparamagnetic, biologically inert, iron-doped yttria-alumina-silica (YAS) glass micro-beads 1 implanted into the intersphincteric plane 3 on either side of a damaged region of tissue 5 in their resting position.

In FIG. 1c, the actuator 9 has been inserted into the anal cavity 7 and the permanent neodymium-iron-boron magnet 11 located within the actuator 9 is shown in a first position about the internal circumference of the actuator 9 close to the micro-beads 1. Operation of the actuator 9 causes the magnet 11 to move around the internal circumference of the actuator 9 thereby causing the micro-beads 1 to move in a circumferential direction in the intersphincteric plane 3 due to magnetic attraction towards the magnet 11. In FIG. 1d, as the magnet 11 moves round the internal circumference of the actuator 9, the distance between the micro-beads 1 and the magnet 11 increases and therefore the magnetic attraction decreases and the micro-beads 1 are restored towards their resting position by the resilience of the internal and external anal sphincter 4, 6.

FIG. 1e shows that as the magnet 11 continues to move around the internal circumference of the actuator 9, the micro-beads are returned to their resting position by the resilience of the internal and external anal sphincter 4, 6.

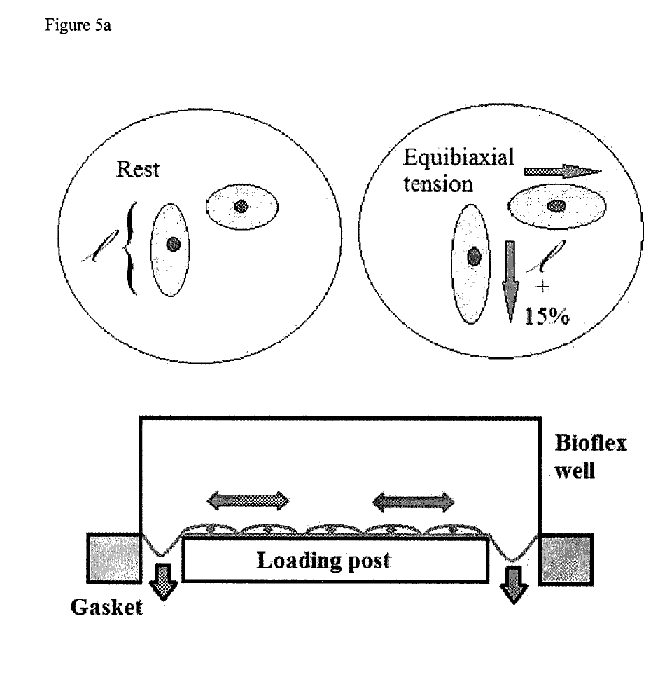

FIG. 2 shows the actuator 15 having a cylindrical, elongate body 17 with rounded, cone shaped tip at the distal end 19 of the actuator 15. A handle portion 21 is formed at the proximal end of the actuator 15 which has a grip 22 and includes a shoulder 23 which extends radially outwardly further the radius of the elongate body 17 in order to prevent over insertion of the actuator 15 into the anal cavity. A cut way shows the rotating member 25 and two permanent magnets 27 attached to the rotating member 25 located within the elongate body 17.

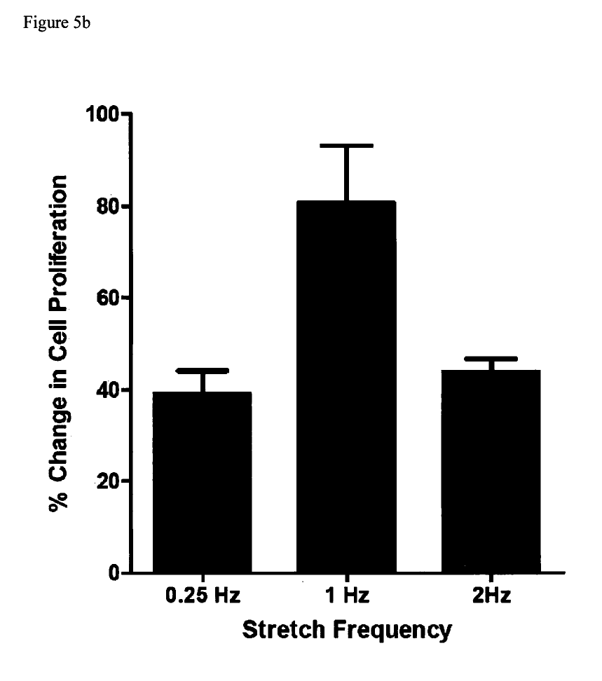

FIG. 3 shows the rotating member 29 having an elongate cylindrical member 31. A portion along part of the length of the cylindrical member 31 has been cut away into which two permanent magnets 33, 35 have been positioned. The magnets 33, 35 do not protrude radially outwards beyond the circumference defined by the cylindrical member 31. The base of the cut away is cover by a sheet of magnetically soft steel 37 on top of which the magnets 33, 35 are mounted. The rotating member also includes, parallel to the length of the cylindrical member 31, a coaxially located axel 39 which is located in the centre of the proximal end 38 of the cylindrical member 31.

FIG. 4 shows the rotating member 41 viewed from the distal end. The end face of the elongate cylindrical body 43 is shown and a rectangular cut away 44 in the side of cylindrical body 43 contains a sheet of magnetically soft steel 45 at the base of the cut away 44. A permanent magnet 47 is mounted on top of the steel sheet 45 and the magnet 47 is oriented such that the magnetic field vector protrudes radially outwards.

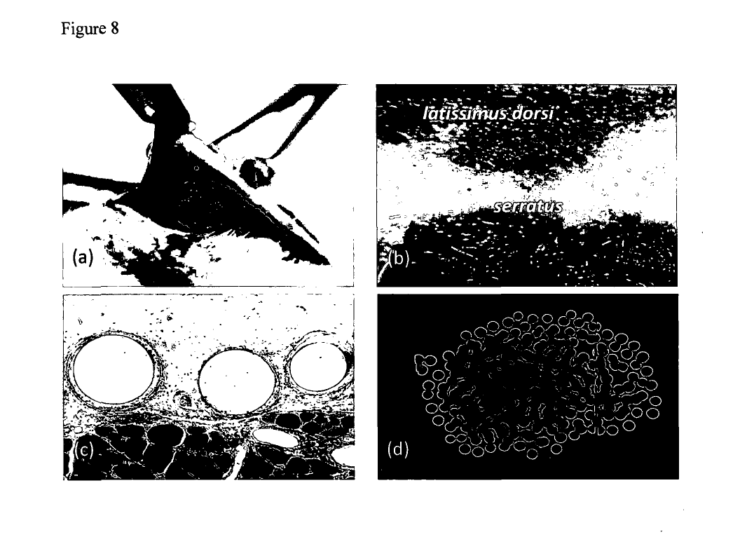

FIG. 8 shows: (a) YAS-Fe₃O₄ glass microspheres implanted into Sprague Dawley rats intermuscularly between the latissimus dorsi and serratus muscles become integrated with host tissue at 3 weeks (b); (c) histology of the excised muscle embedded in resin, with the YAS microspheres etched out using acid, which confirms they become integrated with fibrovascular host tissue; and (d) intermuscularly implanted YAS-Fe₃O₄ microspheres visualised in situ using the SkyScan 1172 micro CT scanner enables 3D evaluation of their distribution pre- and post-magnetic actuation.

### EXAMPLES

### 1. In Vitro Validation of Mechanical Strain Providing a Proliferative Stimulus for Sphincter Muscle Cells

The internal anal sphincter is a specialized continuation of the circular smooth muscle layer of the rectum. Primary cultures of human rectal smooth muscle cells were used as a model system to assess the effect of delivering mechanical strain on cell proliferation. Cells were seeded onto Bioflex® plates and exposed to oscillating mechanical strain at different frequencies using a Flexcell Tension Plus system. Increased proliferation was observed in the cell samples exposed to mechanical strain compared with non-stretched control cells (p<0.0001).

The response of HRSMC to mechanical strain is shown in FIG. 5a. Cells were cultured in wells of a Bioflex® plate. The base of the plate consisted of a silicone membrane that was positioned on a loading post. This allowed the edges of the membrane to be exposed to an oscillating vacuum which generated equibiaxial tension and elongated the membrane by 14%, thus exposing the cells to mechanical strain. FIG. 5b shows the results of exposing the cells to cyclic mechanical strain for 1 hour per day over a 5 day period resulted in a significant increase in cell proliferation (measured by BrdU ELISA) for all frequencies tested compared with non-stretched control cells (p<0.0001).

### 2. In Vitro Validation of Mechanical Strain Delivered Via Magnetic Actuation Stimulates Cell Proliferation

Demonstration that mechanical strain delivered to HRSMC via magnetic actuation increases cell proliferation. Primary cultures of HRSMCs were seeded onto Bioflex® plates, which were customized by the addition of ferromagnetic material to the underside of the silicone membrane. Cyclic mechanical strain was delivered to the cells by positioning a rotating permanent magnet beneath the culture plate. The rotating magnetic field resulted in the base of the Bioflex® plate being stretched and relaxed, resulting in the cells attached to the silicone membrane being exposed to cyclic mechanical strain. Cell proliferation (measured by BrdU ELISA) in HRSMCs exposed to an oscillating magnetic field (0.7 Hz; 1 hour per day for 5 days) was increased by 21±0.05% compared with non-actuated control cells (p<0.05).

### 3. Production of One Embodiment of the Device of the Invention

An embodiment of the invention—a system comprising of a magnetic actuator device and superparamagnetic microspheres has been developed.

The first part of the system consists of superparamagnetic microspheres specifically designed for minimally invasive delivery into the intersphincteric plane. The microspheres have been produced from yttria-alumina-silica (YAS) glass doped with 33 wt % iron oxide (Fe₃O₄) nanoparticles and exhibit a strong response to external magnetic fields.

The YAS-Fe₃O₄ micropsheres have a smooth exterior surface, as shown by light microscopy and scanning elecron microscopy of the microsphere surface. The size range of the microsspheres (212-250 μm) is well above the accepted migratory threshold size (80 μm) for materials implanted into the intersphincteric plane. The microspheres exhibit strong attraction to externally applied magnetic fields, as shown by the placement of a 15 mm diameter×4 mm neodymium iron boron (NdFeB) permanent disc magnet adjacent to a vial of the microspheres.

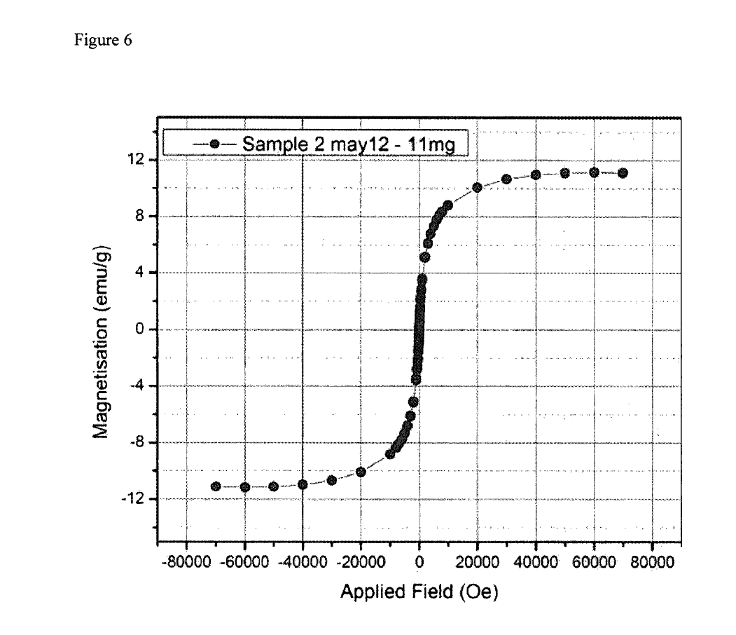

As shown in FIG. 6, the YAS-Fe₃O₄ microspheres are superparamagnetic at room temperature and do not retain any magnetisation after removal of a magnetic field (the magnetic loop is closed rather than open). This enables the microspheres to be free-flowing, which will aid minimally invasive delivery through a syringe and needle, but retain a strong response to an applied magnetic field.

The YAS-Fe₃O₄ microspheres can be implanted between muscle planes, where they exhibit good biocompatibility and integration with host tissue (FIG. 8). Fibrovascular tissue surrounds the implanted microspheres, holding them in position between the muscle bundles. Because the implanted microspheres are superparamagnetic, they will deliver mechanical strain to the host muscle when they are attracted towards an externally applied magnet field.

Delivery of cyclic mechanical strain to the sphincter muscle via the superparamagnetic microspheres implanted into the intersphincteric plane may be achieved using an endoanal device that contains a rotating permanent magnet. An example of an endoanal device according to the invention has been designed with dimensions that will enable the magnetic field from the rotating magnets to cover the length of the sphincter muscle in the anal canal, which is typically ˜25 mm. Two 6 mm×8 mm×12 mm N50 NdFeB magnets, magnetized in opposing directions, are housed in a Delrin rod driven by a motorised spindle. A soft steel plate positioned behind the magnets will throw the magnetic field to the front of the device so that there is effectively zero field except in an ‘arc’ immediately in front of the magnets, confirmed by Vector Fields modelling. This will achieve strong pull and relaxation of the microspheres in the sphincter muscle as the magnet rotates, providing cyclic mechanical strain. The Delrin rod containing the magnets is housed in a Delrin casing that will enable insertion into the anal canal. Vector Fields modelling used to estimate the net force generated by the magnetic field produced with the prototype device on 100 mm³ of the microspheres positioned at a distance equating to the intersphincteric plane equals ˜2 millinewtons.

### 4. Demonstration of Force Delivered to Tissue by the Example System of the Invention

Digital image calibration reveals the prototype endoanal device inserted inside rolled pieces of porcine psoas major muscle (used to simulate the anal canal) delivers mechanical strain to ferromagnetic material placed on the surface of the tissue, simulating positioning of the magnetic microspheres at the intersphincteric plane.

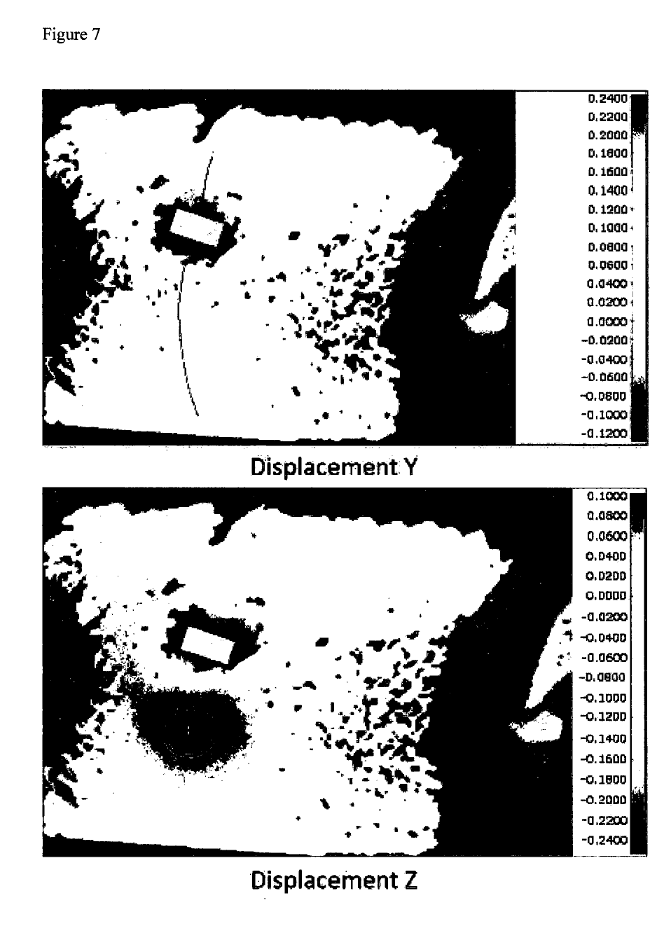

The prototype endoanal device was inserted into rolled pieces of porcine psoas major muscle that were fashioned into a tube by suturing to simulate the anal canal. The thickness of the tissue was 4 mm, approximating the tissue depth from the surface of the anal canal to the intersphincteric plane. The magnets inside the device were rotated with a battery powered motor. Displacement of the ferromagnetic material on the surface of the tissue (*) was measured using a digital image correlation system. The results are shown in FIG. 7.

1. A muscle conditioning device comprising:
one or more implantable bodies; and
an actuator,
where in use, the implantable bodies are magnetically coupled to the actuator and are resiliently positionable to administer mechanical strain to the muscle on operation of the actuator.

2. A device according to claim 1, wherein the muscle is a sphincter.

3. A device according to claim 1 or 2, wherein the muscle is an anal sphincter.

4. A device according any of claims 1 to 3, wherein the implantable bodies are magnetic and the actuator comprises at least one magnet.

5. A device according to any preceding claim, wherein the implantable bodies comprise a magnetic material selected from: iron oxide, ferrites, rare-earth metals, titanium coated neodymium iron boron, samarium cobalt and magnetic steels or a combination thereof.

6. A device according to claim 5, wherein the magnetic material is iron oxide.

7. A device according to any preceding claim, wherein the implantable bodies are magnetic at room temperature or above.

8. A device according to any preceding claim, wherein the implantable bodies comprise an inert coating or inert material.

9. A device according to claim 8, wherein the inert coating or inert material is selected from: gold, titanium, silicone, biocompatible glass, biocompatible polymers including polyesters, polyethylene glycol or a combination thereof.

10. A device according to any preceding claim, wherein the implantable bodies comprise yttria-alumina-silica (YAS) glass.

11. A device according to any preceding claim, wherein the implantable bodies are substantially spherical.

12. A device according to any of preceding claim, wherein the implantable bodies have a diameter in the range of 1 nm to 1 mm.

13. A device according to any of preceding claim, wherein the implantable bodies are nanoparticles.

14. A device according to claim 13, wherein the nanoparticles are positionable within or on the surface cells of a human or animal body.

15. A device according to claim 13 or 14, wherein the nanoparticles comprise a targeting moiety adapted to deliver nanoparticles to a specific cell type.

16. A device according to any of preceding claim, wherein the actuator is operable to generate a magnetic field which creates an oscillating magnetic force on the implantable bodies.

17. A device according to any of preceding claim, wherein the actuator further comprises an elongate body and a rotatable member, wherein the rotatable member is located within the elongate body and is connected to the at least one magnet.

18. A device according to claim 17, wherein the rotatable member rotates about the axis of the elongate body.

19. A device according to claim 17 or 18, wherein the at least one magnet is arranged such that the magnetic field projects radially outwards relative to the axis of rotation.

20. A device according to any of claims 17 to 19, wherein the actuator further comprises a magnetically soft flux concentrator between the at least one magnet and the rotatable member.

21. A device according to any of claims 17 to 20, wherein the actuator further comprises a drive means arranged to rotate the rotatable member.

22. A device according to claim 21, wherein the actuator further comprises a power supply to power the drive means.

23. A device according to any of claims 16 to 22, wherein the actuator further comprises a removable end cap at the distal end of the actuator.

24. A device according to any of claims 16 to 23, wherein the actuator further comprises a handle portion at the proximal end of the actuator.

25. A device according to claim 24, wherein the handle portion further comprises a shoulder arranged to prevent the actuator from being over inserted into the body.

26. A non-totipotent cell comprising the one or more implantable bodies, wherein the one or more implantable bodies are magnetic nanoparticles.

27. A composition comprising a cell according to claim 26 or one or more implantable bodies comprising a magnetic material and a pharmaceutically acceptable excipient.

28. A composition comprising:
one or more implantable bodies; and
a pharmaceutically acceptable excipient,
wherein the one or more implantable bodies are magnetic nanoparticles comprising a targeting moiety adapted to deliver the nanoparticles to a specific cell type.

29. A composition comprising:
one or more implantable bodies; and
a pharmaceutically acceptable excipient,
wherein the implantable bodies have a diameter in the range of 1 μm to 1 mm and comprise a magnetic material and an inert coating or inert material.

30. A composition according to claim 29, wherein the inert coating or inert material is selected from: gold, titanium, silicone, biocompatible glass, biocompatible polymers including polyesters, polyethylene glycol or a combination thereof.

31. A composition according to claim 29 or 30, wherein the implantable bodies comprise yttria-alumina-silica (YAS) glass.

32. A composition according to any of claim 27 or claim 31, wherein the excipient has a viscosity sufficient to form a homogeneous suspension with the implantable bodies.

33. A kit comprising the composition according to any of claims 27 to 32 and an applicator arranged to implant the one or more implantable bodies.

34. An actuator operable to generate a magnetic field which creates an oscillating magnetic force on an implantable body, comprising
an elongate body;
a rotatable member comprising at least one magnet; and
a magnetically soft flux concentrator between the at least one magnet and the rotatable member,
wherein the rotatable member is located within the elongate body and is rotatable about the axis of the elongate body and the at least one magnet is arranged such that the magnetic field projects radially outwards relative to the axis of rotation.

35. A device according to any of claims 1 to 25 for use in the treatment and/or prevention of faecal incontinence.

36. A method of conditioning a muscle using the device according to any of claims 1 to 25 comprising the steps of:
1) positioning one or more implantable bodies to administer mechanical strain to the muscle on operation of the actuator;
2) positioning the actuator in communication with the implantable body; and
3) operating the actuator to administer mechanical strain to the muscle.

37. A method of treating and/or preventing faecal incontinence using the device according to any of claims 1 to 25 comprising the steps of:
1) positioning the implantable bodies in the intersphincteric plane;
2) inserting the actuator into the anal cavity; and
3) operating the actuator to administer mechanical strain to the anal sphincter.

38. A device substantially as described herein with reference to the accompanying description and drawings.
